(19) United States
(12) Patent Application Publication
(10) Pub. No.: US 2022/0125349 A1
(43) Pub. Date: Apr. 28, 2022

(54) NON-INVASIVE SENSING ELECTRODE FOR DETERMINING CONCENTRATION OF GLUCOSE IN LIQUID SAMPLE AND METHOD FOR MANUFACTURING THE SAME

(71) Applicant: National Tsing Hua University, Hsinchu City (TW)

(72) Inventors: Nyan-Hwa TAI, Hsinchu City (TW); Chi-Young LEE, Hsinchu City (TW); Ching CHANG, Hsinchu City (TW); Jia-Ying LIN, Hsinchu City (TW); Shau-San WU, New Taipei City (TW); Jin-Liang CHEN, New Taipei City (TW); You-Ti KUO, New Taipei City (TW)

(73) Assignee: National Tsing Hua University, Hsinchu City (TW)

(21) Appl. No.: 17/146,040

(22) Filed: Jan. 11, 2021

(30) Foreign Application Priority Data
Oct. 26, 2020 (TW) 109137045

(51) Int. Cl. A61B 5/145; G01N 27/30; G01N 27/22

(52) U.S. Cl. CPC A61B 5/14532; G01N 27/308; A61B 2562/164; A61B 2562/125; G01N 27/226

(57) ABSTRACT

A non-invasive sensing electrode for determining a concentration of glucose in a liquid sample includes a conductive substrate body, a composite layer disposed on the conductive substrate body, and a modifying layer disposed on the composite layer. The composite layer includes a plurality of carbon nanotubes randomly crossing one another, and a plurality of gold nanoparticles attached randomly to the carbon nanotubes. The modifying layer includes a plurality of reduced graphene oxide nanowebs separately attached to the composite layer.

### CROSS-REFERENCE TO RELATED APPLICATION

This application claims priority of Taiwanese Invention Patent Application No. 109137045, filed on Oct. 26, 2020.

### FIELD

The disclosure relates to a non-invasive sensing electrode, and more particularly to a non-invasive sensing electrode for determining a concentration of glucose in a liquid sample. The disclosure also relates to a method for manufacturing the non-invasive sensing electrode.

### BACKGROUND

With changes in dietary culture, diabetes is now a metabolic disease of great concern to countries around the world. Monitoring blood glucose level has become a clinically recognized and widely used test for diagnosis and treatment of diabetes. Therefore, in order to avoid health risk or increased medical burden, regular monitoring of blood glucose level is an important therapeutic and preventive practice for diabetic patients and potential diabetic patients.

However, the current mainstream methods for monitoring blood glucose level are all based on an invasive way for collecting blood (for example, by pricking a finger of a subject). This invasive way will not only cause pain to the patient, but will also make the patient resisting the test. In addition, the wound caused by pricking will cause discomfort and create bruises to the patient, and even the risks of the patient fainting and developing wound infection, which further add confusion and hindrance to the measurement process.

Therefore, development of a non-invasive and user-friendly blood glucose monitoring device for self-detecting of blood glucose level at home will benefit many diabetic patients and potential diabetic patients.

### SUMMARY

Therefore, a first object of the disclosure is to provide a non-invasive sensing electrode for determining a concentration of glucose in a liquid sample, and specifically to provide a non-invasive sensing electrode for determining a concentration of glucose in saliva.

A second object of the disclosure is to provide a method for manufacturing the non-invasive sensing electrode.

According to a first aspect of the disclosure, there is provided a non-invasive sensing electrode for determining a concentration of glucose in a liquid sample, such as saliva. The non-invasive sensing electrode includes a conductive substrate body, a composite layer disposed on the conductive substrate body, and a modifying layer disposed on the composite layer. The composite layer includes a plurality of carbon nanotubes randomly crossing one another, and a plurality of gold nanoparticles attached randomly to the carbon nanotubes. The modifying layer includes a plurality of reduced graphene oxide nanowebs separately attached to the composite layer.

A liquid sample, such as saliva, of a subject is mixed with an enzyme, such as glucose oxidase (GOx), to prepare a test liquid. The test liquid is then contacted with the non-invasive electrode according to the disclosure to determine a concentration of glucose in the liquid sample.

According to a second aspect of the disclosure, there is provided a method for manufacturing a non-invasive sensing electrode for determining a concentration of glucose in a liquid sample. The method includes the steps of:

a) preparing a conductive substrate unit, which includes a conductive substrate body and a binder layer disposed on the conductive substrate body;

b) preparing a composite solution including a plurality of carbon nanotubes and a plurality of gold nanoparticles attached randomly to the carbon nanotubes;

c) mixing a portion of the composite solution with graphene oxide to prepare a modifying solution;

d) applying the composite solution on the conductive substrate unit;

e) applying the modifying solution on the composite solution to form a semi-product; and

f) heating the semi-product to remove the binder layer and to partially reduce the graphene oxide to reduced graphene oxide so as to obtain the non-invasive sensing electrode.

In the non-invasive sensing electrode according to the disclosure, the carbon nanotubes of the composite layer randomly cross one another so that the composite layer is provided with a high specific surface area for contacting the liquid sample, and the gold nanoparticles of the composite layer are attached randomly to the carbon nanotubes so that the non-invasive sensing electrode according to the disclosure have superior sensitivity. In addition, the modifying layer includes the reduced graphene oxide nanowebs separately attached to the composite layer, so that the composite layer can be protected by the modifying layer to prevent the composite layer from shedding, and so that the non-invasive sensing electrode according to the disclosure may have a superior conductivity. Furthermore, it is not necessary to coat the enzyme on the non-invasive sensing electrode according to the disclosure, thereby reducing the production cost for manufacturing the non-invasive sensing electrode according to the disclosure, and avoiding the disadvantage of insufficient mixing between the liquid sample and the enzyme in a sensing electrode that is coated with the enzyme.

### BRIEF DESCRIPTION OF THE DRAWINGS

Other features and advantages of the disclosure will become apparent in the following detailed description of the embodiments with reference to the accompanying drawings, of which:

FIG. 1 is a schematic view of an embodiment of an non-invasive sensing electrode for determining a concentration of glucose in a liquid sample according to the disclosure;

FIG. 2 is a schematic view of a composite layer included in the embodiment of the non-invasive sensing electrode according to the disclosure;

FIG. 3 is a schematic perspective view illustrating consecutive steps of an embodiment of a method for manufacturing a non-invasive sensing electrode for determining a concentration of glucose in a liquid sample according to the disclosure;

FIG. 4 is a transmission electron microscopic (TEM) image of the composite layer;

FIG. 5 is a scanning electron microscopic (SEM) image of a modifying layer included in the embodiment of the non-invasive sensing electrode according to the disclosure;

FIG. 6 shows cyclic voltammographs for a conductive substrate body, a laminate of the conductive substrate body and a layer of polyaniline, a laminate of the conductive substrate body and a layer of carbon nanotubes, and a laminate of the conductive substrate body and the composite layer, respectively;

FIG. 7 shows cyclic voltammographs obtained by measuring redox currents of various liquid samples using the embodiment of the non-invasive sensing electrode according to the disclosure;

FIG. 8 is a plot showing a relationship between current difference and glucose concentration;

FIG. 9 shows comparison of a cyclic voltammograph of a liquid sample containing glucose to that of a liquid sample containing a mixture of glucose and ascorbic acid, which are obtained by measuring redox currents with use of the embodiment of the non-invasive sensing electrode according to the disclosure;

FIG. 10 shows comparison of a cyclic voltammograph of a liquid sample containing glucose to that of a liquid sample containing a mixture of glucose and dopamine, which are obtained by measuring redox currents with use of the embodiment of the non-invasive sensing electrode according to the disclosure; and

FIG. 11 shows comparison of a cyclic voltammograph of a liquid sample containing glucose to that of a liquid sample containing a mixture of glucose and uric acid, which are obtained by measuring redox currents with use of the embodiment of the non-invasive sensing electrode according to the disclosure.

### DETAILED DESCRIPTION

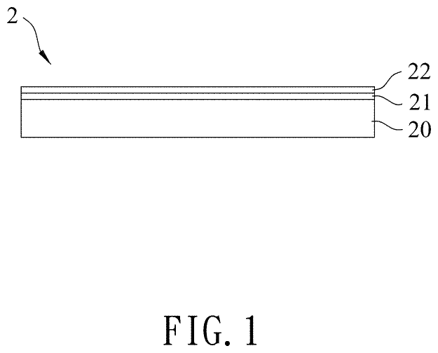

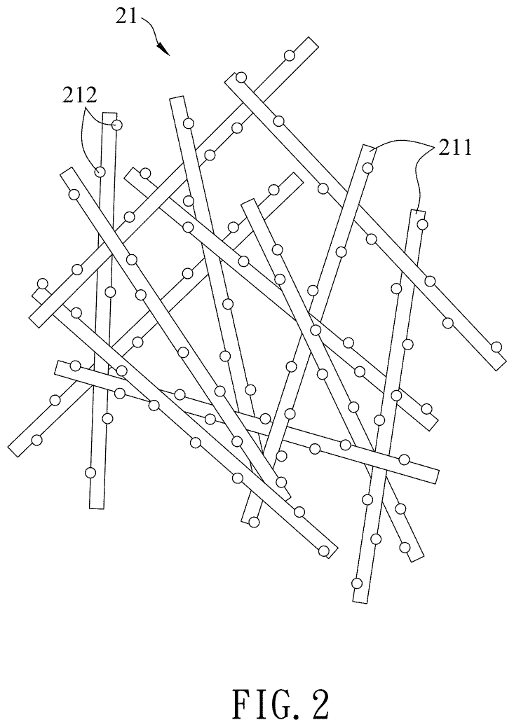

Referring to FIGS. 1 and 2, an embodiment of a non-invasive sensing electrode 2 according to the disclosure is used for determining a concentration of glucose in a liquid sample of a subject, and is specifically used for determining a concentration of glucose in saliva, sweat, tears, or the like of the subject.

The non-invasive sensing electrode 2 includes a conductive substrate body 20, a composite layer 21 disposed on the conductive substrate body 20, and a modifying layer 22 disposed on the composite layer 21.

The conductive substrate body 20 may be a fluorine-doped tin oxide (FTO) substrate, an indium tin oxide (ITO) substrate, a glassy carbon substrate, or combinations thereof. In the embodiment, a FTO substrate is used as the conductive substrate body 20.

The composite layer 21 includes a plurality of carbon nanotubes (CNTs) 211 randomly crossing one another, and a plurality of gold nanoparticles 212 attached randomly to the carbon nanotubes 211.

The modifying layer 22 includes a plurality of reduced graphene oxide nanowebs separately attached to the composite layer 21.

When the non-invasive sensing electrode 2 is used for determining a concentration of glucose in a liquid sample (e.g., saliva, sweat, or tears), the liquid sample is mixed with an enzyme, such as glucose oxidase (GOx), to prepare a test liquid. The test liquid is then subjected to contact with the non-invasive sensing electrode 2 so as to determine the concentration of glucose in the liquid sample.

In the non-invasive sensing electrode 2, the carbon nanotubes 211 of the composite layer 21 randomly cross one another so that composite layer 21 is provided with a high specific surface area for contacting the liquid sample, and the gold nanoparticles 212 of the composite layer 21 are attached randomly to the carbon nanotubes 211, so that the non-invasive sensing electrode 2 have superior sensitivity.

In addition, the modifying layer 22 includes the reduced graphene oxide nanowebs separately attached to the composite layer 21, so that the composite layer 21 can be protected by the modifying layer 22 to prevent the composite layer 21 from shedding and so that the non-invasive sensing electrode 2 may have a superior conductivity.

Furthermore, it is not necessary to coat an enzyme on the non-invasive sensing electrode 2 according to the disclosure, thereby reducing the production cost for manufacturing the non-invasive sensing electrode according to the disclosure, and avoiding the disadvantage of insufficient mixing between the liquid sample and the enzyme in a sensing electrode coated with the enzyme.

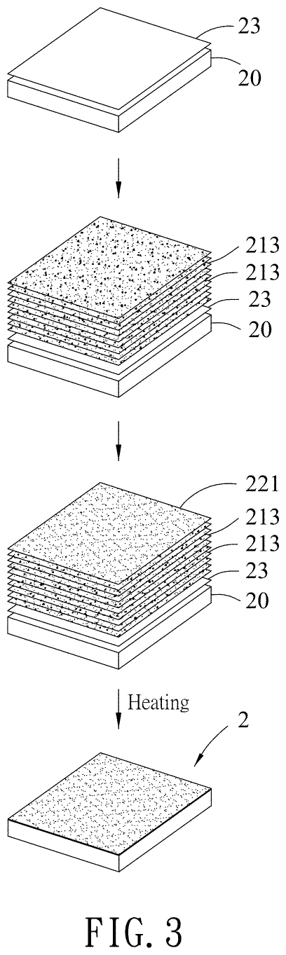

Referring to FIGS. 1 to 3, an embodiment of a method for manufacturing the non-invasive sensing electrode 2 includes the steps of:

a) preparing a conductive substrate unit, which includes the conductive substrate body 20 and a binder layer 23 disposed on the conductive substrate body 20;

b) preparing a composite solution including a plurality of the carbon nanotubes 211 and a plurality of the gold nanoparticles 212 attached randomly to the carbon nanotubes 211;

c) mixing a portion of the composite solution with graphene oxide to prepare a modifying solution 221;

d) applying the composite solution on the conductive substrate unit;

e) applying the modifying solution 221 on the composite solution to form a semi-product; and

f) heating the semi-product to remove the binder layer 23 and to partially reduce the graphene oxide to reduced graphene oxide so as to obtain the non-invasive sensing electrode 2 which includes the conductive substrate body 20, the composite layer 21 disposed on the conductive substrate body 20, and the modifying layer 22 disposed on the composite layer 21, as shown in FIG. 1. Specifically, the composite layer 21 is formed from the composite solution, and the modifying layer 22 is formed from the modifying solution 221.

In step a), as described above, the conductive substrate body 20 may be a FTO substrate, an ITO substrate, a glassy carbon substrate, or combinations thereof. In the embodiment, a FTO substrate is used as the conductive substrate body 20.

In certain embodiments, the binder layer 23 is a layer of a conductive polymer which is formed on the conductive substrate body 20 by chemical polymerization. In certain embodiments, the binder layer 23 is made of polyaniline (PANI). The binder layer 23 is used to enhance the binding of the conductive substrate body 20 of the conductive substrate unit to the material subsequently applied on the conductive substrate body 20.

In certain embodiments, step b) is implemented by the sub-steps of:

b1) adding the carbon nanotubes 211 to a reducing agent solution including a reducing agent to obtain a dispersion of the carbon nanotubes 211 in the reducing agent solution;

b2) heating the dispersion of the carbon nanotubes 211 in the reducing agent solution to an elevated temperature of at least 100° C. to form a preparative solution; and

b3) adding a gold precursor to the preparative solution at the elevated temperature to subject the gold precursor to a reduction process with the reducing agent so as to form the gold nanoparticles 212 that are attached randomly to the carbon nanotubes 211, thereby obtaining the composite solution.

In certain embodiments, the reducing agent is sodium citrate.

In certain embodiments, in sub-step b2), the dispersion of the carbon nanotubes 211 in the reducing agent solution is heated to 100° C.

In certain embodiments, in sub-step b3), the gold precursor is chloroauric acid (HAuCl₄. 3H₂O).

In certain embodiments, the carbon nanotubes 211 are subjected to an acid treatment prior to sub-step b1) so as to enhance hydrophilicity thereof. In certain embodiments, the acid treatment is implemented by adding the carbon nanotubes 211 to an acid liquid to form a dispersion of the carbon nanotubes 211 in the acid liquid, heating the dispersion of the carbon nanotubes 211 in the acid liquid to an elevated temperature ranging from 70° C. to 100° C., and then cooling down and neutralizing the dispersion of the carbon nanotubes 211 in the acid liquid with deionized water, followed by drying the carbon nanotubes 211 treated with the acid liquid.

In certain embodiments, the acid liquid is a mixture of nitric acid and sulfuric acid.

In certain embodiments, the dispersion of the carbon nanotubes 211 in the acid liquid is heated to 80° C. for 1 hour.

In step c), a portion of the composite solution is mixed with the graphene oxide in a suitable predetermined ratio which may be adjusted according to specific practice to prepare the modifying solution. In certain embodiments, a portion of the composite solution is mixed with the graphene oxide in a predetermined ratio such that the modifying solution thus prepared contains 5 vol % of the graphene oxide.

In certain embodiments, in step d), the composite solution is applied on the binder layer 23 of the conductive substrate unit by drop casting. In certain embodiment, the composite solution are repeatedly applied in a manner that after the composite solution applied previously is dried to form a composite sub-layer 213, the composite solution is again applied on the thus formed composite sub-layer 213.

In certain embodiments, in step f), the semi-product is heated to an elevated temperature ranging from 400° C. to 500° C. When the semi-product is heated to an elevated temperature higher than 500° C., the carbon nanotubes 211 may be destroyed.

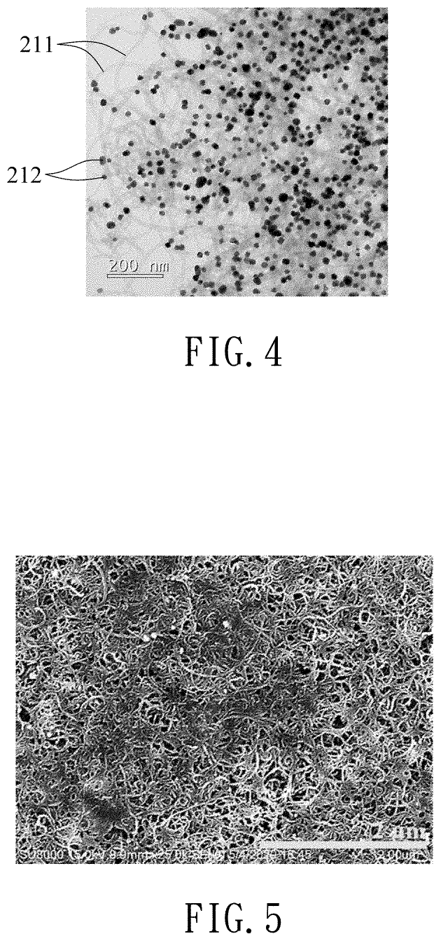

FIG. 4 shows a TEM image of the composite layer 21, in which the carbon nanotubes 211 randomly cross one another and the gold nanoparticles 212 are attached randomly to the carbon nanotubes 211, so that composite layer 21 is provided with a high specific surface area for contacting the liquid sample and so that the non-invasive sensing electrode 2 have superior sensitivity due to improved electron transfer efficiency.

FIG. 5 shows an SEM image of the modifying layer 22. Since the reduced graphene oxide nanowebs of the modifying layer 22 are separately attached to the composite layer 21, an overall specific surface area of the non-invasive sensing electrode 2 is increased effectively, and the composite layer 21 is protected by the modifying layer 22 to prevent the composite layer from shedding. In addition, since the reduced graphene oxide has superior conductivity compared to the graphene oxide, the non-invasive sensing electrode 2 may be conferred with a superior conductivity.

It should be noted that if the modifying solution 221 is prepared merely using the graphene oxide, rather than a mixture of the composite solution and the graphene oxide, a continuous layer of the reduced graphene oxide, rather than a plurality of the reduced graphene oxide nanowebs, are formed on the composite layer 21, so that the overall specific surface area of the non-invasive sensing electrode 2 is decreased, which may reduce the overall conductivity and sensitivity of the non-invasive sensing electrode 2.

When the non-invasive sensing electrode 2 is used for determining a concentration of glucose in a liquid sample (e.g., saliva, sweat, tears, or the like), the liquid sample is mixed with an enzyme, such as glucose oxidase (GOx) to prepare a test liquid. The test liquid is then contacted with the non-invasive sensing electrode 2 to determine the concentration of glucose in the liquid sample.

Since it is not necessary to coat the enzyme on the non-invasive electrode sensing 2, the production cost for manufacturing the non-invasive sensing electrode 2 can be reduced. In addition, the liquid sample is mixed with the enzyme to prepare the test liquid, which is then contacted with the non-invasive sensing electrode 2 to determine the concentration of glucose in the liquid sample. Therefore, the liquid sample can be evenly mixed with the enzyme, so that the disadvantage of insufficient mixing between the liquid sample and the enzyme in a sensing electrode coated with the enzyme can be avoided and so that the sensing performance of the non-invasive sensing electrode 2 can be enhanced.

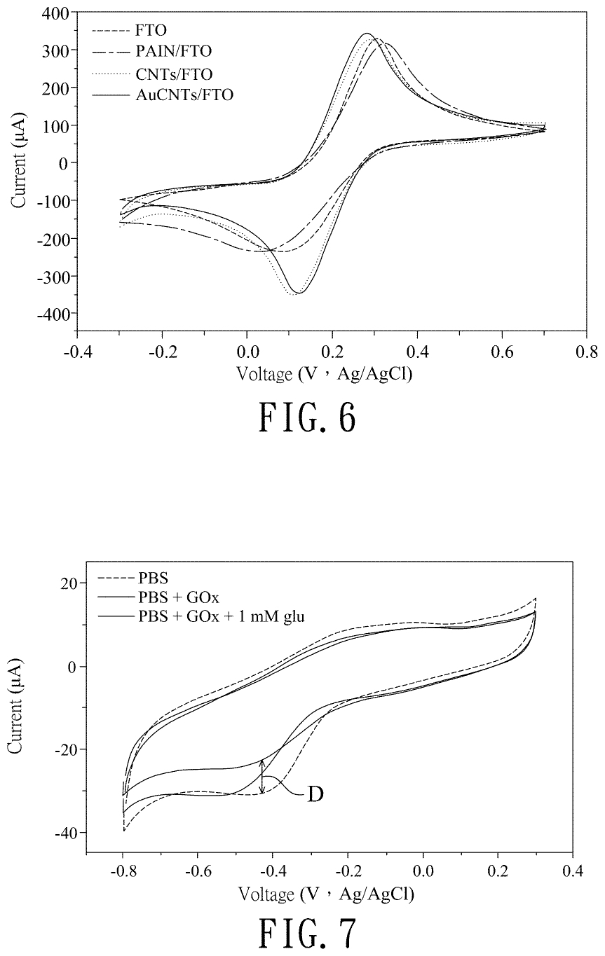

FIG. 6 and Table 1 below respectively show cyclic voltammographs and charge-transfer resistances (R_{ct}) of various components.

The cyclic voltammographs were obtained by performing cyclic voltammetry measurement using a 0.1 M KCl electrolyte solution containing 5.0 mM K₃[Fe(CN)₆] under a voltage window ranging from −0.3 V to 0.7 V, and a scan rate of 0.1 mV/s.

The charge-transfer resistance was obtained by performing electrochemical impedance spectroscopy (EIS) using an electrochemical instrument (Autolab PGSTAT30 & FRA2), which was connected to an electrode system that includes the non-invasive sensing electrode 2, an Ag/AgCl reference electrode, and a platinum auxiliary electrode.

As shown in FIG. 6, a redox peak current of a laminate of the conductive substrate body 20 and the composite layer 21 (referred to as AuCNTs/FTO in FIG. 6) is superior to that of the conductive substrate body (referred to as FTO in FIG. 6).

As shown in Table 1 below, the charge-transfer resistance of the non-invasive sensing electrode 2 (referred to as rGO/AuCNTs/FTO in Table 1) is reduced significantly, indicating that the non-invasive sensing electrode 2 has enhanced electrochemical properties so that the electron transfer efficiency can be improved.

TABLE 1

| | Charge-transfer |
|---|---|
| | resistance |
| | (R_{ct}, Ω) |
| FTO¹ | 3664 |
| PANI²/FTO | 280 |
| AuCNTs³/FTO | 50 |
| rGO⁴/AuCNTs/FTO | 46.5 |

¹FTO: a conductive substrate body of fluorine-doped tin oxide;
²PANI: a binder layer of polyaniline;
³AuCNTs: a composite layer of carbon nanotubes and gold nanoparticles attached randomly to the carbon nanotuves; and
⁴rGO: a modifying layer of reduced graphene oxide.

FIG. 7 shows the cyclic voltammographs obtained by measuring redox currents of various liquid samples using the non-invasive sensing electrode 2. Such liquid samples include phosphate buffered saline (PBS), a mixture of PBS and glucose oxidase (GOx), and a mixture of PBS, GOx, and glucose (glu).

The cyclic voltammographs are obtained by performing cyclic voltammetry measurement under a voltage window ranging from −0.8 V to 0.2 V and a scan rate of 0.1 mV/s. The cyclic voltammetry measurement was first performed using PBS for 15 cycles so as to stabilize the non-invasive sensing electrode 2.

Determination of a glucose concentration in a liquid sample is based on an electrochemical process. The reaction mechanism of the electrochemical process is shown below.

GOx(Cofₒₓ)+Glucose→GOx(Cof_{red})+Gluconic acid GOx(Cof_{red})+O₂→GOx(Cofₒₓ)+H₂O₂

Reduction peaks in the cyclic voltammographs of the various liquid samples are measured by cyclic voltammetry. The reduction peak in the cyclic voltammograph of each of the liquid samples is determined based on a reduction peak current of an oxidation reaction for each of the liquid samples. Specifically, the higher the glucose concentration in the liquid sample, the greater the amount of oxygen being reacted, so that the reduction peak thus obtained is lowered. A current difference (D) between the reduction peak current of the liquid sample containing glucose and that of the test sample of PBS is obtained to determine a sensing response of the non-invasive sensing electrode 2.

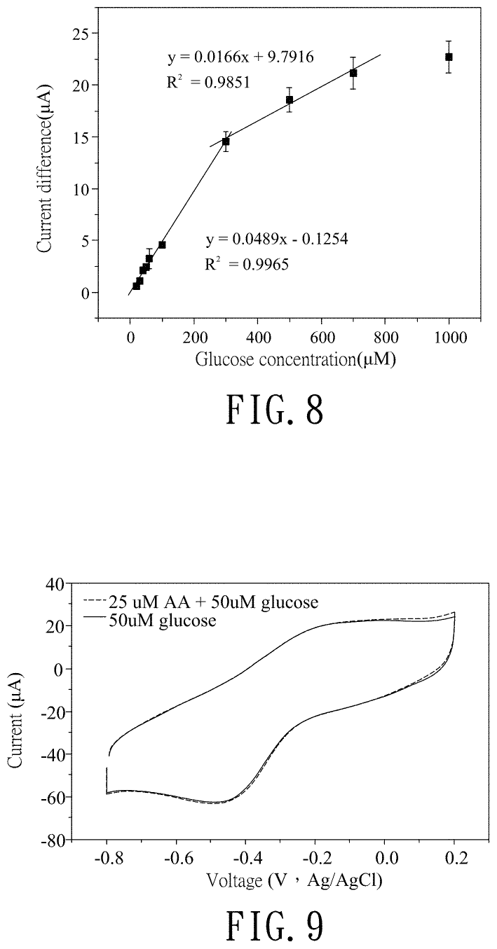

FIG. 8 shows a relationship between the current difference and the glucose concentration. When the glucose concentration in the liquid sample is increased, the current difference is increased due to the reduction peak being lowered. Two linear sensing responses are shown in FIG. 8, one of which is obtained when the glucose concentration ranges from 20 μm to 300 μm (R²=0.9965) with a sensitivity of 127.06 μA/mMcm², and the other one of which is obtained when the glucose concentration ranges from 300 μm to 700 μm (R²=0.9851) with a sensitivity of 43.13 μA/mMcm². The glucose concentration ranging from 20 μm to 700 μm can be determined by the non-invasive sensing electrode 2.

A fasting blood glucose level of a healthy human ranges from 70 to 110 mg/dL (i.e., from 3.89 mM to 6.11 mM), which corresponds to a saliva glucose concentration ranging from 38.9 μM to 61.1 μM. One of the diagnostic criteria for diabetes is that the fasting blood glucose level should be greater than 126 mg/dL (i.e., 7 mM), which can be converted to a saliva glucose concentration of 70 μM. If the saliva glucose concentration exceeds 70 μM, the patient will be in high risk of diabetes. The linear sensing response of the non-invasive sensing electrode 2 is obtained when the glucose concentration ranges from 20 μm to 300 μm, which fully cover the saliva glucose concentration of both healthy people and that of diabetics, indicating that the non-invasive sensing electrode 2 presents great application potential in diagnosis of diabetes.

Human body fluids contain not only glucose but also many other biochemical compounds, such as ascorbic acid (AA), dopamine (DA), and uric acid (UA), which may interfere a glucose response signal during the electrochemical process for determining the glucose concentration. Therefore, a sensing electrode having a superior selectivity for determining the glucose concentration in a liquid sample, such as saliva, is important for accurate measurement of a glucose response signal.

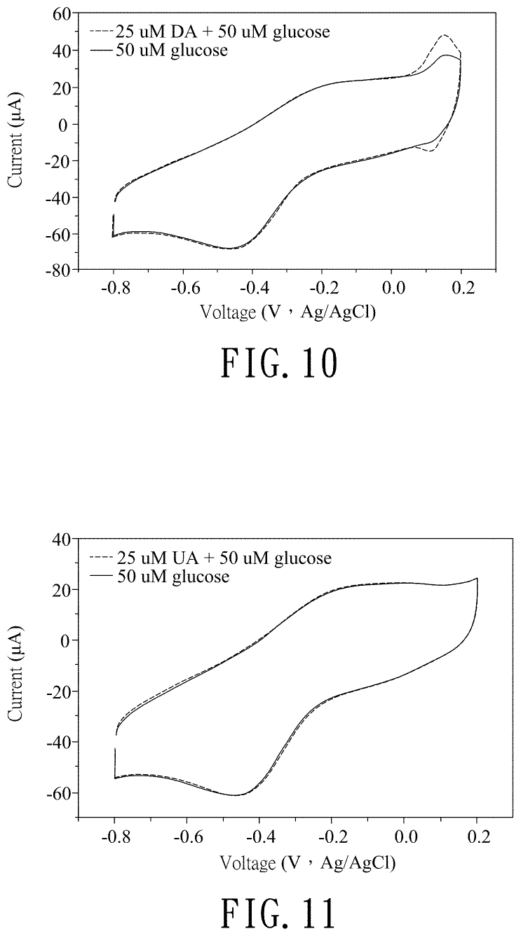

FIGS. 9 to 11 show the results of selectivity tests of different biochemical compounds, which are ascorbic acid (AA), dopamine (DA) , and uric acid (UA), respectively.

The cyclic voltammographs of a 50 μM glucose solution, and a mixture solution containing a 50 μM glucose solution and 25 μM of one of the biochemical compounds were obtained by performing cyclic voltammetry measurement, and are shown in FIGS. 9 to 11.

As shown in FIGS. 9 to 11, none of these biochemical compounds interfere with the glucose response signal. The results indicate that the non-invasive sensing electrode 2 has good selectivity and specificity to glucose and possesses a great potential for determining the concentration of glucose in a liquid sample, such as saliva.

In view of the aforesaid, in the non-invasive sensing electrode according to the disclosure, the carbon nanotubes of the composite layer randomly cross one another so that composite layer is provided with a high specific surface area for contacting the liquid sample, and the gold nanoparticles of the composite layer are attached randomly to the carbon nanotubes, so that the non-invasive sensing electrode according to the disclosure have superior sensitivity. In addition, the modifying layer includes the reduced graphene oxide nanowebs separately attached to the composite layer, so that the composite layer can be protected by the modifying layer to prevent the composite layer from shedding, and so that the non-invasive sensing electrode according to the disclosure may have a superior conductivity. Furthermore, it is not necessary to coat an enzyme on the non-invasive sensing electrode according to the disclosure, thereby reducing the production cost for manufacturing the non-invasive sensing electrode according to the disclosure, and avoiding the disadvantage of insufficient mixing between a liquid sample and the enzyme in a sensing electrode that is coated with the enzyme.

In the description above, for the purposes of explanation, numerous specific details have been set forth in order to provide a thorough understanding of the embodiments. It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A non-invasive sensing electrode for determining a concentration of glucose in a liquid sample, comprising:
a conductive substrate body;
a composite layer disposed on said conductive substrate body, and including a plurality of carbon nanotubes randomly crossing one another and a plurality of gold nanoparticles attached randomly to said carbon nanotubes; and
a modifying layer disposed on said composite layer, and including a plurality of reduced graphene oxide nanowebs separately attached to said composite layer.

2. The non-invasive sensing electrode according to claim 1, wherein said conductive substrate body is selected from the group consisting of a fluorine-doped tin oxide substrate, an indium tin oxide substrate, a glassy carbon substrate, and combinations thereof.

3. A method for manufacturing a non-invasive sensing electrode for determining a concentration of glucose in a liquid sample, comprising the steps of:
a) preparing a conductive substrate unit, which includes a conductive substrate body and a binder layer disposed on the conductive substrate body;
b) preparing a composite solution including a plurality of carbon nanotubes and a plurality of gold nanoparticles attached randomly to the carbon nanotubes;
c) mixing a portion of the composite solution with graphene oxide to prepare a modifying solution;
d) applying the composite solution on the conductive substrate unit;
e) applying the modifying solution on the composite solution to form a semi-product; and
f) heating the semi-product to remove the binder layer and to partially reduce the graphene oxide to reduced graphene oxide so as to obtain the non-invasive sensing electrode.

4. The method according to claim 3, wherein in step a), the binder layer is a layer of a conductive polymer which is formed on the conductive substrate body by chemical polymerization.

5. The method according to claim 3, wherein in step d), the composite solution is applied on the binder layer of the conductive substrate unit by drop casting.

6. The method according to claim 3, wherein step b) includes sub-steps of:
b1) adding the carbon nanotubes to a reducing agent solution including a reducing agent to obtain a dispersion of the carbon nanotubes in the reducing agent solution;
b2) heating the dispersion of the carbon nanotubes in the reducing agent solution to an elevated temperature of at least 100° C. to form a preparative solution; and
b3) adding a gold precursor to the preparative solution at the elevated temperature to subject the gold precursor to a reduction process with the reducing agent so as to form gold nanoparticles attached randomly to the carbon nanotubes.

7. The method according to claim 6, further comprising prior to sub-step b1), a sub-step of subjecting the carbon nanotubes to an acid treatment.

8. The method according to claim 7, wherein the acid treatment is implemented by adding the carbon nanotubes to an acid liquid to form a dispersion of the carbon nanotubes in the acid liquid, heating the dispersion of the carbon nanotubes in the acid liquid to an elevated temperature ranging from 70° C. to 100° C., neutralizing the dispersion of the carbon nanotubes in the acid liquid with deionized water, and drying the carbon nanotubes treated with the acid liquid.

9. The method according to claim 8, wherein the acid liquid is a mixture of nitric acid and sulfuric acid.

10. The method according to claim 3, wherein in step d), the composite solution are repeatedly applied in a manner that after the composite solution applied previously is dried to form a composite sub-layer, the composite solution is again applied on the composite sub-layer.

11. The method according to claim 3, wherein in step f), the semi-product is heated at a temperature ranging from 400° C. to 500° C.

12. The method according to claim 3, wherein the binder layer is made of polyaniline.

13. The method according to claim 6, wherein the reducing agent is sodium citrate, and the gold precursor is chloroauric acid.
